SEQUENCE LISTING (11 sequences)

SEQ ID NO: 1 - DNA, 21 bp, Artificial Sequence
  Description of Artificial Sequence Synthetic primer
ggcaccacaccttctacaatg

SEQ ID NO: 2 - DNA, 20 bp, Artificial Sequence
  Description of Artificial Sequence Synthetic primer
gtggtggtgaagctgtagcc

SEQ ID NO: 3 - DNA, 21 bp, Artificial Sequence
  Description of Artificial Sequence Synthetic primer
ctttggaccctctgacttgag

SEQ ID NO: 4 - DNA, 22 bp, Artificial Sequence
  Description of Artificial Sequence Synthetic primer
ttccacatctatgccacttgag

SEQ ID NO: 5 - DNA, 19 bp, Artificial Sequence
  Description of Artificial Sequence Synthetic primer
tcagtgtggaggtgctttg

SEQ ID NO: 6 - DNA, 21 bp, Artificial Sequence
  Description of Artificial Sequence Synthetic primer
cagccttcccataaggatgaa

SEQ ID NO: 7 - DNA, 22 bp, Artificial Sequence
  Description of Artificial Sequence Synthetic primer
ggagccttcgatcaggtataaa

SEQ ID NO: 8 - DNA, 21 bp, Artificial Sequence
  Description of Artificial Sequence Synthetic primer
ctcaagtcagagggtccaaag

SEQ ID NO: 9 - DNA, 22 bp, Artificial Sequence
  Description of Artificial Sequence Synthetic primer
gaggccaaatttctgctcattg

SEQ ID NO: 10 - DNA, 21 bp, Artificial Sequence
  Description of Artificial Sequence Synthetic primer
gttctttcaggaagcccgtta

SEQ ID NO: 11 - protein, 7 aa, Artificial Sequence
  Description of Artificial Sequence Synthetic peptide
YSPTSPS

(19) United States
(12) Patent Application Publication
(10) Pub. No.: US 2021/0338615 A1
(43) Pub. Date: Nov. 4, 2021

(54) Inhibition of Acetyl-CoA Metabolism for Treatment and Prevention of Immune System Diseases and Disorders

(71) Applicant: Memorial Sloan Kettering Cancer Center, New York, NY (US)

(72) Inventors: Ming Li, New York, NY (US); Min Peng, New York, NY (US); Na Yin, New York, NY (US)

(21) Appl. No.: 17/372,272

(22) Filed: Jul. 9, 2021

Related U.S. Application Data
(62) Division of application No. 16/333,417, filed on Mar. 14, 2019, now abandoned.
(60) Provisional application No. 62/394,859, filed on Sep. 15, 2016.

(51) Int. Cl. A61K 31/192; A61P 37/06; A61K 9/00; A61K 31/10

(52) U.S. Cl. CPC A61K 31/192; A61P 37/06; A61K 31/10; A61K 9/0053; A61K 9/0019

(57) ABSTRACT

Provided are methods for treating or preventing T cell-mediated diseases or disorders by administering an inhibitor of acetyl-CoA production, such as an inhibitor of lactate dehydrogenase A or an inhibitor of ATP-citrate lyase.

### CROSS REFERENCE TO RELATED APPLICATIONS

This application is a divisional of U.S. application Ser. No. 16/333,417, filed on Mar. 14, 2019, which is the National Stage of International Application No. PCT/US2017/051724, filed on Sep. 15, 2017, which claims the benefit of priority of U.S. Provisional Patent Application No. 62/394,859, filed on Sep. 15, 2016, the entire contents of which are incorporated by reference.

### STATEMENT OF GOVERNMENT SUPPORT

This invention was made with government support under grant number CA008748, awarded by the National Institutes of Health. The government has certain rights in the invention.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### BACKGROUND

T cell activation and differentiation are associated with metabolic rewiring (1-4). A metabolic hallmark of activated T cells is aerobic glycolysis (the Warburg effect) (5), the conversion of glucose to lactate in the presence of oxygen. However, its physiopathological functions remain incompletely understood (6-8). As the major carbon source, glucose plays important roles in T cell development, proliferation, and function (9-15). Aerobic glycolysis has been implicated in augmenting effector T cell responses, including expression of the pro-inflammatory cytokine interferon (IFN)-γ, via 3′ untranslated region (3′UTR)-mediated mechanisms. However, the specific contribution of aerobic glycolysis to T cell responses has not been well defined. Using galactose as a sugar source, aerobic glycolysis was proposed to support IFN-γ expression through 3′UTR-mediated mechanisms (12). Although galactose is metabolized at a slower rate than glucose via the Leloir pathway, both sugars are converted to lactate (16), rendering the galactose system unable to model aerobic glycolysis deficiency in a definitive manner.

By converting pyruvate to lactate with regeneration of nicotinamide adenine dinucleotide (NAD⁺) (17), lactate dehydrogenase (LDH) defines the biochemical reaction of aerobic glycolysis. LDHA and LDHB form five tetrameric LDH isoenzymes (A₄B₀, A₃B₁, A₂B₂, A₁B₃, and A₀B₄) with distinct kinetic properties (17). ATP-citrate lyase (ACL) is an enzyme converting citrate to acetyl-CoA in the cytosol.

A better understanding of LDH and ACL activity upon T cell activation could have therapeutic implications for diseases and disorders of the immune system, including autoimmunity and transplant rejection.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The inventors show herein that LDHA is induced in activated T cells to support aerobic glycolysis, but promotes IFN-γ expression independently of its 3′UTR. Instead, LDHA maintains high levels of acetyl-CoA to enhance histone acetylation and transcription of Ifng. Ablation of LDHA in T cells protects mice from immunopathology triggered by excessive IFN-γ expression or deficiency of regulatory T cells. These findings reveal an epigenetic mechanism by which aerobic glycolysis promotes effector T cell differentiation, and indicate that LDHA and downstream enzymes such as ACL can be targeted therapeutically in diseases or disorders associated with T cell activation and/or differentiation and/or proliferation, such as autoinflammatory and autoimmune diseases, graft rejection, and transplant rejection.

Accordingly, the invention provides a method of treating or preventing a T cell-mediated disease or disorder, the method comprising reducing acetyl-CoA production in a subject having or susceptible to developing a T cell-mediated disease or disorder.

In one aspect, the invention provides a method of treating or preventing a T cell-mediated disease or disorder, the method comprising administering to a subject having or susceptible to developing a T cell-mediated disease or disorder a composition comprising an effective amount of a lactate dehydrogenase A (LDHA) inhibitor. In another aspect, the invention provides a composition comprising an LDHA inhibitor for use in the treatment of a T cell-mediated disease or disorder. The invention also provides a composition comprising an LDHA inhibitor for the manufacture of a medicament for use in the treatment of a T cell-mediated disease or disorder. In some embodiments, the LDHA inhibitor is selected from the group consisting of FX11, Galloflavin, GNE-140, GSK 2837808A, and NHI 2. In a preferred embodiment, the LDHA inhibitor is FX11.

In a further aspect, the invention provides a method of treating or preventing a T cell-mediated disease or disorder, the method comprising administering to a subject having or susceptible to developing a T cell-mediated disease or disorder a composition comprising an effective amount of an ATP-citrate lyase (ACL) inhibitor. In yet another aspect, the invention provides a composition comprising an ACL inhibitor for use in the treatment of a T cell-mediated disease or disorder. The invention also provides a composition comprising an ACL inhibitor for the manufacture of a medicament for use in the treatment of a T cell-mediated disease or disorder. In some embodiments, the ACL inhibitor is selected from the group consisting of BMS 303141, ETC-1002, and SB 204990. In a preferred embodiment, the ACL inhibitor is BMS 303141.

In some embodiments, the composition is administered orally. In some embodiments, the composition is administered intravenously.

In a preferred embodiment, the subject is a human.

T cell-mediated diseases or disorders that can be treated or prevented by the methods of the invention include, but are not limited to, autoimmune disorders, graft rejection, inflammation, and organ rejection. In certain embodiments, the autoimmune disorder is selected from the group consisting of graft-versus-host disease (GVHD), inflammatory bowel disease (IBD), multiple sclerosis (MS), psoriasis, rheumatoid arthritis, systemic lupus erythematosus (SLE), and type-1 diabetes. In one embodiment, the composition is administered prophylactically within about 24 hours of an organ transplant or tissue graft.

### BRIEF DESCRIPTION OF THE FIGURES

FIG. 1 shows that LDHA is upregulated in CD4+ T cells upon TCR stimulation. Panel A shows zymography of LDH isoenzymes in muscle, heart, and activated CD4⁺ T cells (T cell), 4 LDHA and 0 LDHB (A₄B₀), A₃B₁, A₂B₂, A₁B₃, and A₀B₄. Panel B shows naïve CD4⁺ T cells isolated from wild-type (WT) or CD4^{Cre}Ldha^{fl/fl} (KO) mice, left untreated or stimulated with anti-CD3 and anti-CD28 in the presence of IL-2 for 3 days. The expression of LDHA, LDHB, and GAPDH was determined by immunoblotting with tissues from muscle and heart as controls. Asterisk indicates the LDHB band recognized by the cross-reactive anti-LDHA.

FIG. 2 shows that LDHA dictates aerobic glycolysis in activated CD4+ T cells. Naïve CD4⁺ T cells isolated from wild-type (WT) or CD4^{Cre}Ldha^{fl/fl} (KO) mice were stimulated with anti-CD3 and anti-CD28 in the presence of IL-2 for 2 days. Cells were replenished with fresh medium, which was harvested 24 h later. Lactate production (panel A) and glucose consumption (panel B) were determined with triplicates. Naïve WT or KO CD4⁺ T cells were stimulated as in panel A. Extracellular acidification rate (ECAR) and oxygen consumption rate (OCR) were measured with a glycolysis stress test kit (panels C, E, and F) or a Mito stress test kit (panels D, G-I). Baseline ECAR (panel E) and stressed ECAR (panel F) of activated CD4⁺ T cells were calculated according to panel C. Baseline OCR (panel G), stressed OCR (panel H), and baseline OCR/ECAR (panel I) were calculated according to panel D. Statistics (panels E-I) were from one of two independent experiments, each with 8 biological replicates (n=8); data represent mean±SD, two-tailed unpaired t-test, ***P≤0.001.

FIG. 3 shows metabolic flux analysis of wild-type (WT) and LDHA-deficient CD4+ T cells. Naïve CD4⁺ T cells isolated from WT or CD4^{Cre}Ldha^{fl/fl} (KO) mice were differentiated under Th1 conditions for 3 days. Cells were labeled with ¹³C₆-glucose (10 mM) in glucose-deficient RPMI1640 supplemented with 10% dialyzed FBS and 100 U/mL IL-2 for 2 h. Metabolites were extracted by methanol and analyzed by CE-MS. Data represent mean±SD, n=3. Two-tailed unpaired t-test, *P≤0.05; **P≤0.01; ***P≤0.001; ****P≤0.0001.

FIG. 4 shows that LDHA deficiency does not affect T cell development in mice. Panel A shows flow cytometry analysis of CD4 and CD8 expression in thymocytes (left panels), and Foxp3 expression in CD4⁺CD8⁻ T cells (right panels) of 8-week-old wild-type (WT) or CD4^{Cre}Ldha^{fl/fl} (KO) mice. Panels B and C show frequencies of CD4⁻CD8⁻ (DN), CD4⁺CD8⁺ (DP), CD4⁺CD8⁻ (CD4SP), and CD4⁻CD8⁺ (CD8SP) thymocytes (panel B, n=7) and Foxp3⁺ regulatory T cells among CD4SP cells (panel C, n=5). Data represent mean±SD. Two-tailed unpaired t-test; ns, not significant.

FIG. 5 shows that LDHA deficiency does not substantially affect T cell homeostasis in mice. Panel A shows total peripheral lymph node (pLN) and spleen (SPL) cell numbers from wild-type (WT) and CD4^{Cre}Ldha^{fl/fl} (KO) mice. Data represent mean±SD, n=7 per genotype. Two-tailed unpaired t-test, ns, not significant. Panels B-E show flow cytometry analysis of pLN and SPL T cell populations from WT and KO mice. Panels B and C show representative plots of TCRβ⁺, CD4⁺ TCRβ⁺, CD8⁺ TCRβ⁺, and CD4⁺ TCRβ⁺Foxp3⁺ T cell populations. Panels D and E show frequencies and numbers of TCRβ⁺, CD4⁺ TCRβ⁺, CD8⁺ TCRβ⁺, and CD4⁺ TCRβ⁺Foxp3⁺ T cell populations. Data represent mean±SD, n=7 per genotype. Two-tailed unpaired t-test, ns, not significant. Panels F and G show flow cytometry analysis of CD44 and CD62L expression in pLN and SPL CD4⁺ TCRβ⁺ or CD8⁺ TCRβ⁺ T cells. Panel F shows representative plots of CD44 and CD62L expression. Panel G shows frequencies of CD4⁺ (upper panel) and CD8⁺ (lower panel) T cell populations with the indicated activation phenotypes. Data represent mean±SD, n=8 per genotype. Two-tailed unpaired t-test, *P≤0.05; ns, not significant.

FIG. 6 shows that LDHA is non-essential for CD4+ T cell activation, growth, proliferation, and survival in vitro. Panels A-D show naïve CD4⁺ T cells isolated from wild-type (WT) or CD4^{Cre}Ldha^{fl/fl} (KO) mice, and stimulated with anti-CD3 and anti-CD28 in the presence of IL-2. Panel A shows flow cytometry analysis of CD44, CD69, and CD25 expression one day post-activation, and CD40L expression 3 days post-activation. Panel B shows cell size of WT and KO T cells, measured 3 days post-activation. Panels C and D show flow cytometry analysis of DAPI staining of WT and KO T cells 3 days post-activation. Panel C shows representative plots of DAPI staining. Panel D shows frequencies of DAPI⁻ T cells, n=4. Two-tailed paired t-test, ns, not significant. Panel E shows naïve CD4⁺ WT and KO T cells labeled with CFSE and stimulated as in panel A. CFSE dilution was examined 1, 2, and 3 days post-stimulation. Data are representative of three independent experiments.

FIG. 7 shows that LDHA regulates IFN-γ expression independent of its 3′ UTR. Panels A and B show naïve wild-type (WT) or CD4^{Cre}Ldha^{fl/fl} (KO) CD4⁺ T cells differentiated under Th1 conditions for 3 days, and re-stimulated with phorbol myristate acetate (PMA) and ionomycin for 4 h. IFN-γ expression was determined by intracellular staining. Representative plots (panel A) and mean fluorescence intensity (MFI) (panel B) are shown. Panels C-E show naïve WT or KO CD4⁺ T cells activated for 2 days and transduced with green fluorescent protein (GFP) constructs fused with the 3′ untranslated region (3′UTR) of the Ifng or Gapdh gene. GFP expression was measured 48 h after transduction. Representative plots (panel C) and MFI of GFP in GFP⁺ cells (panel D) are shown. Panel E shows MFI of GFP from IFN-γ 3′UTR, normalized to that of GAPDH 3′UTR control. Panel F shows a diagram of IFN-γ mRNA expressed from the WT Ifng or Yeti allele. Panels G and H show naïve CD4⁺ T cells from Yeti/Yeti or KOYeti/Yeti mice, differentiated as in panel A. The expression of IFN-γ and eYFP were determined by flow cytometry. Representative plots and respective statistical analysis are shown in panels G and H. Statistics (panels B, E, and H) were from one of two independent experiments, each with 3 biological replicates (n=3); data represent mean±SD, two-tailed unpaired t-test, *P≤0.05; ***P≤0.001; ns, not significant.

FIG. 8 shows genome-wide analysis of LDHA-regulated transcripts in Th1 cells. Panel A shows a volcano plot of transcripts differentially expressed between wild-type (WT) and CD4^{Cre}Ldha^{fl/fl} (KO) T cells, differentiated under Th1 conditions. Arrow indicates the differentially expressed Ifng gene. FDR corrected p value <0.01. Panel B shows a scatter plot of relative histone acetylation (H3K9Ac) levels of the differentially expressed transcripts between WT and KO Th1 cells. Arrow indicates the differential H3K9Ac at the promoter region of the Ifng gene. FDR corrected p value <0.01. Panel C shows a selected list of genes with either decreased or increased level of mRNA (heat map) and H3K9Ac (black box) in KO Th1 cells compared to those of WT Th1 cells.

FIG. 9 shows that IFN-γ but not T-bet expression is diminished in LDHA-deficient Th1 cells. Naïve wild-type (WT) or CD4^{Cre}Ldha^{fl/fl} (KO) CD4⁺ T cells were differentiated under Th1 conditions for 3 days. Panel A shows IFN-γ mRNA measured by qPCR and normalized to I3-Actin. Panels B and C show T-bet expression measured by flow cytometry. Representative plots (panel B) and statistics (panel C) are shown. In panels A and C, data represent mean±SD, n=3. Two-tailed unpaired t-test, ***P≤0.001; ns, not significant.

FIG. 10 shows that LDHA promotes IFN-γ expression through an epigenetic mechanism. Panel A shows representative H3K9 acetylation (H3K9Ac) peaks at the Cd3e or Ifng promoter and enhancer (CNS22) regions from one of two ChIP-Seq experiments. Panel B shows H3K9Ac at the Ifng promoter and CNS22 enhancer regions in WT or KO Th1 cells, assessed by ChIP-qPCR. Enrichment was normalized to H3K9Ac at the Cd3e promoter region. Panel C shows naïve WT or KO CD4⁺ T cells differentiated under Th1 conditions, and acetyl-CoA levels, measured by an acetyl-CoA assay kit. Panels D and E show naïve WT or KO CD4⁺ T cells differentiated under Th1 conditions for 3 days, and either left untreated or supplemented with 20 mM sodium acetate for another 24 h. Cells were subsequently re-stimulated with phorbol myristate acetate (PMA) and ionomycin for 4 h. The expression of IFN-γ protein (panel D) and mRNA (panel E) were determined by flow cytometry and qPCR, respectively. mRNA level of IFN-γ was normalized to that of (3-Actin. MFIs of IFN-γ are shown in panel D. Panel F shows T cells cultured as in panel D. H3K9Ac at the Ifng promoter and CNS22 enhancer regions were assessed by ChIP-qPCR. Enrichment was normalized to H3K9Ac at the Cd3e promoter region. Statistics (Panels B, C, E, and F) were from one of three independent experiments, each with 3 biological replicates (n=3); data represent mean±SD, two-tailed unpaired t-test, *P≤0.05; **P≤0.01; ***P≤0.001; ns, not significant.

FIG. 11 shows that acetylation of histone H3 and recruitment of RNA polymerase II to the Ifng locus are reduced in LDHA-deficient Th1 cells. Naïve wild-type (WT) or CD4^{Cre}Ldha^{fl/fl} (KO) CD4⁺ T cells were differentiated under Th1 conditions for 3 days, and subjected to ChIP with a control antibody (IgG) or antibodies against total histone H3 (panel A), acetylated lysine 27 of histone H3 (H3K27Ac) (panel B), or RNA polymerase II (PolII) (panel C). Enrichment was normalized to the Cd3e promoter region. Data represent mean±SD, n=3. Two-tailed unpaired t-test, *P≤0.05; **P≤0.01; ***P≤0.001; ns, not significant.

FIG. 12 shows that an ACL inhibitor suppresses IFN-γ expression, while acetate and an HDAC inhibitor promote IFN-γ expression. In panels A-D, naïve CD4⁺ T cells isolated from wild-type (WT) mice were differentiated under Th1 conditions for 2 days. DMSO or the ATP citrate lyase inhibitor (ACLi) BMS-303141 was added to the culture for another 24 h. Subsequently, cells were re-stimulated with phorbol myristate acetate (PMA) and ionomycin for 4 h. IFN-γ expression was determined by intracellular staining. A representative plot (panel A) and statistical analysis (panel B) are shown. Cell viability was determined by LIVE/DEAD staining. A representative plot (panel C) and statistical analysis (panel D) are shown. Data represent mean±SD, n=3. Two-tailed unpaired t-test, *P≤0.05; **P≤0.01; ns, not significant. Panel E shows naïve CD4^{Cre}Ldha^{fl/fl} (KO) CD4⁺ T cells differentiated under Th1 conditions for 2 days. Cells were replated in fresh medium with or without 20 mM sodium acetate for another 24 h. Acetyl-CoA levels were measured by an acetyl-CoA assay kit. Data represent mean±SD, n=3. Two-tailed unpaired t-test, **P≤0.01. Panel F shows naïve CD4⁺ T cells isolated from WT or KO mice, differentiated under Th1 conditions for 3 days. Cells were further cultured in the absence or presence of 20 mM sodium acetate for another 24 h, followed by re-stimulation with PMA and ionomycin for 4 h. The expression of T-bet was determined by flow cytometry. Panels G and H show naïve CD4⁺ T cells isolated from WT or KO mice, differentiated under Th1 conditions for 3 days. Cells were further cultured in the absence or presence of 30 nM TSA for another 24 h, followed by re-stimulation with PMA and ionomycin for 4 h. The expression of IFN-γ (panel G) and T-bet (panel H) were determined by flow cytometry. The mean fluorescence intensity (MFI) of IFN-γ is shown in panel G. Panel I shows cells cultured as in panel G. H3K9Ac at the Ifng promoter region was assessed by ChIP-qPCR. Enrichment was normalized to H3K9Ac at the Cd3e promoter region. Data represent mean±SD, n=4. Two-tailed unpaired t-test, **P≤0.01; ***P≤0.001; ns, not significant.

FIG. 13 shows that the LDHA inhibitor FX11 suppresses IFN-γ expression in T cells. Naïve CD4⁺ T cells isolated from wild-type mice were differentiated under Th1 conditions for 3 days. DMSO or indicated concentration of Fx11 was added to the cell culture for another 24 h, with or without 20 mM sodium acetate. Subsequently, cells were re-stimulated with phorbol myristate acetate (PMA) and ionomycin for 4 h. IFN-γ expression was determined by intracellular staining. Representative plots (panel A) and quantification (panel B) are shown.

FIG. 14 shows that LDHA deficiency in T cells protects Yeti/Yeti mice from a lethal autoinflammatory disease. Panel A shows the survival curve of Yeti/Yeti (n=5) and CD4^{Cre}Ldha^{fl/fl}Yeti/Yeti (KO Yeti/Yeti) mice (n=6). Panel B shows representative haematoxylin and eosin staining of liver sections from wild-type (WT), CD4^{Cre}Ldha^{fl/fl} (KO), Yeti/Yeti, and KO Yeti/Yeti mice. Arrows indicate the necrotic regions. In panels C and D, splenocytes from mice of the indicated genotypes were stimulated with phorbol myristate acetate (PMA) and ionomycin for 4 h. IFN-γ and YFP expression in CD4⁺ T cells and in NK1.1⁺ TCR⁻ NK cells were determined by flow cytometry. Representative plots (Panel C) and statistics (Panel D) are shown; data represent mean±SD, n=4-5 mice per genotype, two-tailed paired t-test, ***P≤0.001; ns, not significant.

FIG. 15 shows that LDHA promotes IFN-γ expression in the absence of its 3′ UTR in vivo. Panel A shows representative images of livers from wild-type (WT), CD4^{Cre}Ldha^{fl/fl} (KO), Yeti/Yeti, and CD4^{Cre}Ldha^{fl/fl}Yeti/Yeti (KO Yeti/Yeti) mice. Arrows indicate the damaged liver in Yeti/Yeti mice. Panels B and C show total cell numbers (panel 5B) and CD4⁺ T cell numbers (panel C) from spleens of WT, KO, Yeti/Yeti, and KO Yeti/Yeti mice. Data represent mean±SD, n=4-5 per genotype. Two-tailed unpaired t-test, ns, not significant. Panels D and E show flow cytometry analysis of CD44 and CD62L expression in CD4⁺ T cells from WT, KO, Yeti/Yeti, and KO Yeti/Yeti mice. Panel D shows representative plots of CD44 and CD62L expression. Panel E shows frequencies of CD4⁺ T cell populations with the indicated activation phenotypes; data represent mean±SD, n=4-5 per genotype. Two-tailed unpaired t-test, *P≤0.05; ns, not significant.

FIG. 16 show that LDHA deficiency in T cells attenuates the autoimmune phenotype of Scurfy mice. Panel A shows a representative image of 21-day-old wild-type (WT), CD4^{Cre}Ldha^{fl/fl} (KO), Foxp3sf, and CD4^{Cre}Ldha^{fl/fl}Foxp3sf (KO Foxp3sf) mice. Arrows indicate the scurfy tails and ears of Foxp3sf mice, which were rescued in KO Foxp3sf mice. Panel B shows the survival curve of Foxp3sf (n=25) and KO Foxp3sf (n=16) mice. Panel C shows representative haematoxylin and eosin (H&E) staining of sections from the lung, liver, and ear of WT, KO, Foxp3sf, and KO Foxp3sf mice. Panel D shows a representative image of peripheral lymph nodes and spleens of 21-day-old WT, KO, Foxp3sf, and KO Foxp3sf mice. Panels E and F show total cell numbers (panel E) and CD4⁺ T cell numbers (panel F) from peripheral lymph nodes (pLN) and spleens (SPL) of WT, KO, Foxp3sf, and KO Foxp3sf mice; data represent mean±SD, n=4-9 per genotype. Two-tailed unpaired t-test, *P≤0.05; ns, not significant. Panels G and H show flow cytometry analysis of CD44 and CD62L expression in CD4⁺ T cells from WT, KO, Foxp3sf, and KO Foxp3sf mice. Panel G shows representative plots of CD44 and CD62L expression. Panel H shows frequencies of CD4⁺ T cell populations with the indicated activation phenotypes; data represent mean±SD, n=4-9 per genotype. Two-tailed unpaired t-test, *P≤0.05; ***P≤0.001; ns, not significant. Panels I and J show CD4⁺ T cells from peripheral pLN or spleens SPL of WT, KO, Foxp3sf, and KO Foxp3sf mice, stimulated with PMA and ionomycin for 4 h. IFN-γ expression was determined by flow cytometry. Representative plots (panel I) and paired analyses (panel J) are shown. Data represent mean±SD, n=2-7 per genotype. Two-tailed paired t-test, *P≤0.05; ***P≤0.001; ns, not significant.

FIG. 17 shows that LDHA deficiency results in compromised Th17 cell differentiation, which can be recapitulated by pharmacologic inhibition of LDHA with an LDHA inhibitor (GNE-140). Naïve WT or LDHA-deficient (KO) CD4⁺ T cells were differentiated under Th17 conditions for 4 days. Increasing doses of GNE-140 were also included in the WT T cell culture. Cell viability was determined by a live/dead cell dye, and the percentage of live cell population is shown. In addition, cells were re-stimulated with PMA/ionomycin for 4 h. IL-17a and Foxp3 protein expression was examined by flow cytometry, and used as readouts for Th17 and Treg cell differentiation, respectively.

FIG. 18 shows that compromised Th17 cell differentiation in the absence of LDHA is associated with reduced H3K9Ac, and that inhibition of acetyl-CoA generation by an ACL inhibitor (BMS303141) protects mice from experimental autoimmune encephalomyelitis (EAE), a mouse model of multiple sclerosis. In panels A-C, naïve WT or LDHA-deficient (KO) CD4⁺ T cells were differentiated under Th17 conditions for 3 days. Cells were re-stimulated with PMA/ionomycin for 4 h. Panel A shows IL-17a protein expression examined by flow cytometry. Panel B shows IL-17a mRNA expression measured by qPCR, and normalized to that of β-actin. Data represent mean±SD, n=2. Two-tailed unpaired t-test, **P≤0.01. Panel C shows representative H3K9Ac peaks at the Cd3e and 1117a loci from one of two ChIP-Seq experiments. In panels D-F, WT or KO mice were subjected to EAE induction by immunization with MOG peptide with CFA. Clinical scoring in panel D: 0, no overt signs of disease; 1, limp tail; 2, partial hind-limb paralysis; 3, total hind-limb paralysis; 4, hind-limb paralysis and 75% body paralysis; 5, complete body paralysis, or moribund state, or death by EAE, or sacrifice for humane reasons. Panel E shows immune cells from spinal cord and cerebellum, re-stimulated with PMA/ionomycin for 4 h. IL-17a and IFN-γ protein expression were examined by flow cytometry. Panel F is quantification of panel E; data represent mean±SD, n=4. Two-tailed unpaired t-test, ***P≤0.001. Panel G shows that an ACL inhibitor (ACLi, BMS 303141) inhibits Th17 cell differentiation in vitro. Naïve WT or KO CD4⁺ T cells were differentiated under Th17 conditions for 3 days, and BMS 303141 was included in the culture for another 24 hours. Cells were re-stimulated with PMA/ionomycin for 4 h. IL-17a protein expression was examined by flow cytometry. Panel H shows that BMS 303141 treatment protects mice from EAE. WT mice were subjected to EAE induction by immunization with MOG peptide with CFA. BMS 303141 (35 mg/kg/day) was intraperitoneally injected daily, starting from day 7. Clinical scoring was the same as in panel D.

FIG. 19 shows an epigenetic model of aerobic glycolysis in the control of IFN-γ expression. A major role of LDHA-mediated aerobic glycolysis in activated CD4⁺ T cells is to maintain glycolytic production of ATP, which relieves the burden of mitochondria as an energy house to burn acetyl-CoA to generate ATP. As a result, acetyl-CoA can be readily used as a substrate for histone acetyltransferases (HATs) for epigenetic regulation of target genes including Ifng. In the absence of LDHA, glucose consumption is reduced and more acetyl-CoA is oxidized in the mitochondria to generate ATP, leading to reduced nucleocytosolic acetyl-CoA for histone acetylation and attenuated transcription of target genes.

### DETAILED DESCRIPTION OF THE INVENTION

We demonstrate that genes involved in aerobic glycolysis, such as lactate dehydrogenase A (LDHA), also play a role in T cell differentiation by an epigenetic mechanism, which is distinct from how they were thought to act. Deficiency of this enzyme (or inhibition of the enzyme) reverses the symptoms of immune disorders seen in the Scurfy mouse model. Scurfy mice have defective T cell tolerance leading to an X-linked lymphoproliferative disease that parallels the X-linked autoimmunity-allergic disregulation syndrome (XLAAD) in humans. This work has identified a critical role for LDHA-mediated aerobic glycolysis in promoting autoreactive Th1 and Th17 cell responses. LDHA inhibitors are being developed to target tumor cell metabolism. Our data indicate that LDHA inhibitors are immunosuppressive, and thus may complicate their applications in cancer. Instead, LDHA inhibitors, as well as inhibitors targeting other enzymes involved in acetyl-CoA metabolism, including ACL, can be useful in treatment of diseases and disorders associated with T cell activation, differentiation, or expansion.

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10. Where a numeric term is preceded by “about,” the term includes the stated number and values ±10% of the stated number. The headings provided herein are not limitations of the various aspects or embodiments of the invention, which can be had by reference to the specification as a whole. Accordingly, the terms defined immediately below are more fully defined by reference to the specification in its entirety.

Amino acids are referred to herein by their commonly known three-letter symbols or by the one-letter symbols recommended by the IUPAC-IUB Biochemical Nomenclature Commission. Nucleotides, likewise, are referred to by their commonly accepted single-letter codes. Unless otherwise indicated, amino acid sequences are written left to right in amino to carboxy orientation, and nucleic acid sequences are written left to right in 5′ to 3′ orientation.

Wherever embodiments are described with the language “comprising,” otherwise analogous embodiments described in terms of “consisting of” and/or “consisting essentially of” are included.

The term “T cell-mediated disease or disorder,” as used herein, includes diseases and disorders characterized by T cell activation, T cell differentiation, and/or T cell proliferation. T cell-mediated inflammation, graft or organ rejection, and autoimmune diseases, such as graft-versus-host disease (GVHD), inflammatory bowel disease (IBD), multiple sclerosis (MS), psoriasis, rheumatoid arthritis, systemic lupus erythematosus (SLE), and type-1 diabetes, are examples of T cell-mediated diseases or disorders.

An “active agent” is an agent which itself has biological activity, or which is a precursor or prodrug that is converted in the body to an agent having biological activity. Active agents useful in the methods of the invention include inhibitors of acetyl-CoA production, such as LDHA inhibitors and ACL inhibitors.

An “LDHA inhibitor” is an active agent that agonizes or antagonizes the activity of lactate dehydrogenase A or reduces its production in a cell. Examples of LDHA inhibitors that are suitable for use in the present invention include FX11, Galloflavin, GNE-140, quinoline 3-sulfonamides (38), including GSK 2837808A, and NHI 2. “FX11” refers to a compound having the structure:

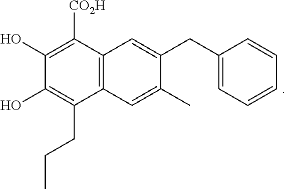 [SMILES: CCCc1c(O)c(O)c(C(=O)O)c2cc(Cc3ccccc3)c(C)cc12]

“Galloflavin” refers to 3,8,9,10-tetrahydroxy-pyrano[3,2-c][2]benzopyran-2,6-dione. “GNE-140” refers to (2R)-5-(2-chlorophenyl)sulfanyl-4-hydroxy-2-(4-morpholin-4-ylphenyl)-2-thiophen-3-yl-1,3-dihydropyridin-6-one. “GSK 2837808A” refers to 3-[[3-(cyclopropylsulfamoyl)-7-(2,4-dimethoxypyrimidin-5-yl)quinolin-4-yl]amino]-5-(3,5-difluorophenoxy)benzoic acid. “NHI 2” refers to methyl 1-hydroxy-6-phenyl-4-(trifluoromethyl)-1H-indole-2-carboxylate. Derivatives of these compounds that act as LDHA inhibitors, and their pharmaceutically acceptable salts, are also suitable for use in the invention.

An “ACL inhibitor” is an active agent that agonizes or antagonizes the activity of ATP-citrate lyase or reduces its production in a cell. Examples of ACL inhibitors that are suitable for use in the present invention include BMS 303141, ETC-1002, MEDICA 16, and SB 204990. “BMS 303141” refers to 3,5-dichloro-2-hydroxy-N-(4-methoxy[1,1′-biphenyl]-3-yl)-benzenesulfonamide. “ETC-1002” refers to 8-hydroxy-2,2,14,14-tetramethylpentadecanedioic acid. “MEDICA 16” refers to 3,3,14,14-tetramethylhexadecanedioic acid. “SB 204990” refers to (3R,5S)-rel-5-[6-(2,4-dichlorophenyl)hexyl]tetrahydro-3-hydroxy-2-oxo-3-furanacetic acid.

The terms “inhibit,” “block,” and “suppress” are used interchangeably and refer to any statistically significant decrease in biological activity, including full blocking of the activity.

By “subject” or “individual” or “patient” is meant any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder.

“Prevent” or “prevention” refers to prophylactic or preventative measures that prevent and/or slow the development of a targeted pathologic condition or disorder. Thus, those in need of prevention include those at risk of or susceptible to developing the disorder. Subjects that are at risk of or susceptible to developing a T cell-mediated disease or disorder include, but are not limited to, patients receiving a tissue graft or an organ transplant, patients having a genetic predisposition to a T cell-mediated disease or disorder, and patients having another disease or disorder of the immune system, including another T cell-mediated disease or disorder. In certain embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

In a prophylactic context, a composition for inhibiting acetyl-CoA production, such as a composition comprising an LDHA inhibitor or an ACL inhibitor, can be administered at any time before or after an event, for example, a tissue graft or an organ transplant, which places a subject at risk of or susceptible to developing a T cell-mediated disease or disorder. In some aspects, the composition for inhibiting acetyl-CoA production is administered prophylactically up to about one week before the event, such as 1, 2, 3, 4, 5, 6, or 7 days before the event. In some instances, the composition for inhibiting acetyl-CoA production is administered prophylactically on the same day as the event. In some embodiments, the pharmaceutical composition is administered prophylactically within 7 days of the event, for example, within about 1, 2, 3, 4, 5, 6, or 7 days. In a preferred embodiment, the composition for inhibiting acetyl-CoA production is administered within 24 hours of the event, i.e., about 24 hours before the beginning of the event to about 24 hours after the completion of the event.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be in numerous dosage forms, for example, tablet, capsule, liquid, solution, softgel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

“Systemic administration” means that a pharmaceutical composition is administered such that the active agent enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous. “Local administration” means that a pharmaceutical composition is administered directly to where its action is desired (e.g., at or near the site of the injury or symptoms). Local routes of administration include, without limitation, topical, inhalational, subcutaneous, ophthalmic, and otic. It is within the purview of one of ordinary skill in the art to formulate pharmaceutical compositions that are suitable for their intended route of administration.

An “effective amount” of a composition as disclosed herein is an amount sufficient to carry out a specifically stated purpose. An “effective amount” can be determined empirically and in a routine manner, in relation to the stated purpose, route of administration, and dosage form.

In some embodiments, administration of the LDHA inhibitor or the ACL inhibitor can comprise systemic administration, at any suitable dose and/or according to any suitable dosing regimen, as determined by one of skill in the art. The LDHA inhibitor or the ACL inhibitor can be administered according to any suitable dosing regimen, for example, where the daily dose is divided into two or more separate doses. It is within the skill of the ordinary artisan to determine a dosing schedule and duration for administration. In some embodiments, the pharmaceutical composition is administered orally at least once a day or at least twice a day. In some embodiments, the pharmaceutical composition is administered intravenously at least once a day or at least twice a day. In some embodiments, the pharmaceutical composition is administered subcutaneously at least once a day or at least twice a day.

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1. Absence of LDHA Alters Glucose Metabolism in Activated CD4+ Cells

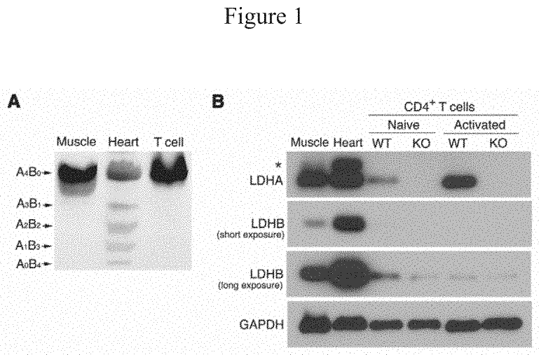

To characterize LDH activity in activated CD4+ cells, we performed a zymography assay. Activated CD4⁺ T cells manifested LDH activity predominantly in the form of A₄B₀, similar to that of muscle tissues (FIG. 1, panel A). Consistently, LDHA, but not LDHB, was induced upon T cell activation (FIG. 1, panel B), likely due to HIF-1α- and c-Myc-induced transcription of Ldha (10, 11).

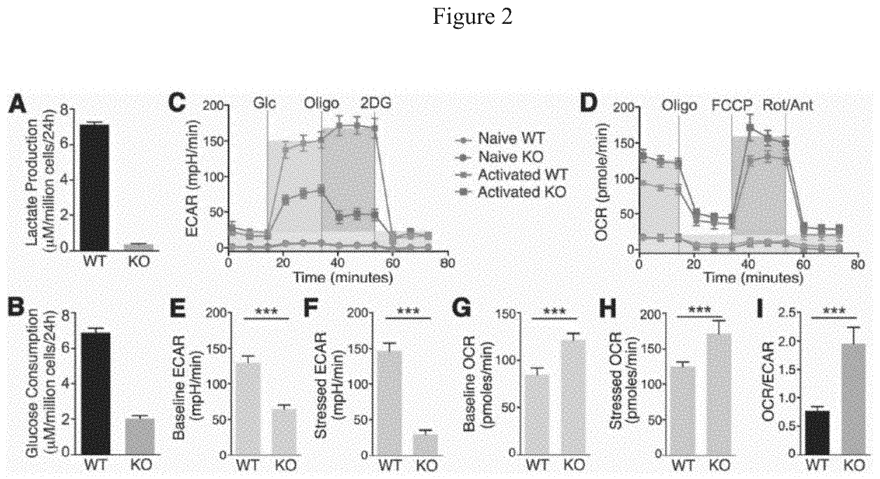

To study the definitive function of aerobic glycolysis, we deleted LDHA specifically in T cells (CD4^{Cre}Ldha^{fl/fl}, designated as knock-out, KO) (FIG. 1, panel B) (18). Compared to activated wild-type (WT) CD4⁺ T cells, KO T cells barely produced lactate (FIG. 2, panel A). Furthermore, glucose consumption in KO T cells was reduced to 30% WT levels (FIG. 2, panel B), in line with a critical role for LDHA in sustaining aerobic glycolysis through regeneration of NAD⁺ consumed at the glyceraldehyde-3-phosphate dehydrogenase (GAPDH) step of glycolysis (17). Consistent with low Glut1 (13) and LDHA expression (FIG. 1, panel B), naïve CD4⁺ T cells showed little activity of glucose metabolism with negligible glucose-induced extracellular acidification rate (ECAR) or oxygen consumption rate (OCR) (FIG. 2, panels C and D). Upon activation, ECAR was increased in WT CD4⁺ T cells, which was largely diminished in KO T cells (FIG. 2, panels C, E, and F). In contrast, both basal and maximal OCR were elevated in KO T cells (FIG. 2, panels D, G, and H).

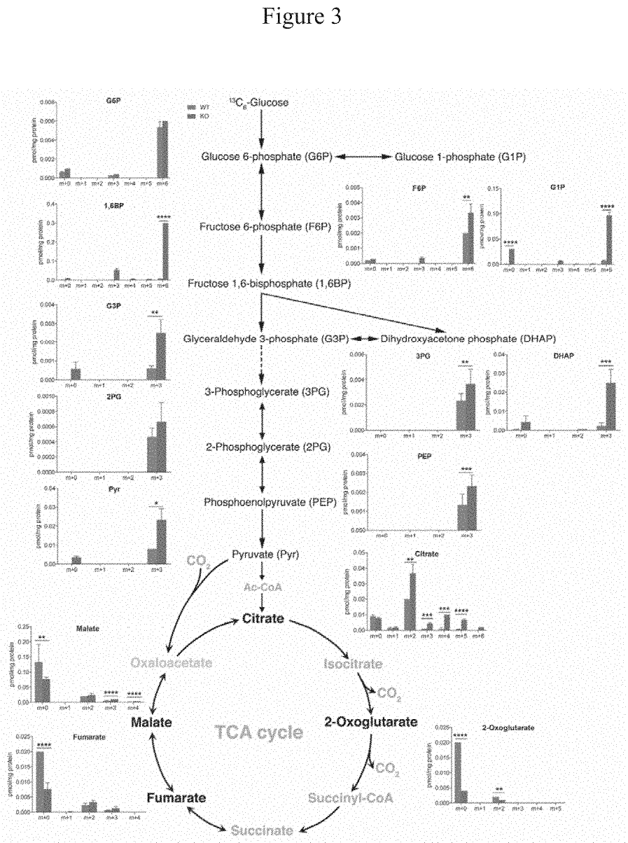

¹³C-isotope labeled glucose (¹³C₆-glucose) tracing experiments showed that glycolysis was slowed down at the GAPDH step in KO T cells (FIG. 3). However, ¹³C-labelled citrate was increased in the absence of LDHA (FIG. 3). In particular, using doubly (m+2) and quadruply (m+4) ¹³C-labeled citrate, readouts of tricarboxylic acid (TCA) cycle activity through the first and second turns, respectively, were much increased in KO T cells (FIG. 3), in line with enhanced OCR (FIG. 2, panels D, G, and H). Together, these findings reveal that glucose metabolism in activated CD4⁺ T cells is shifted from aerobic glycolysis towards oxidative phosphorylation in the absence of LDHA (FIG. 2, panel I).

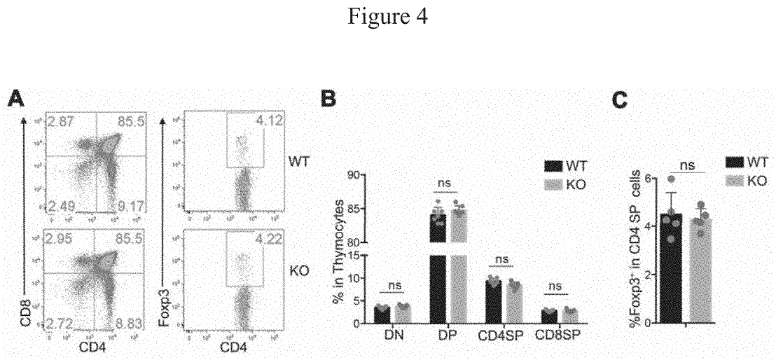

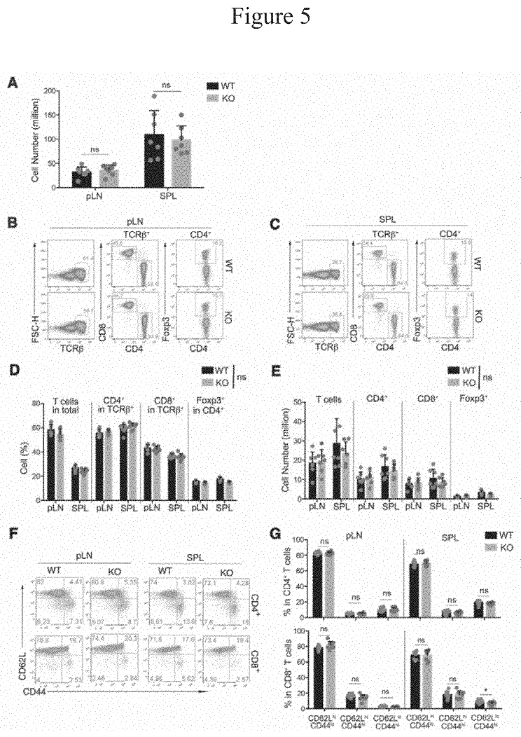

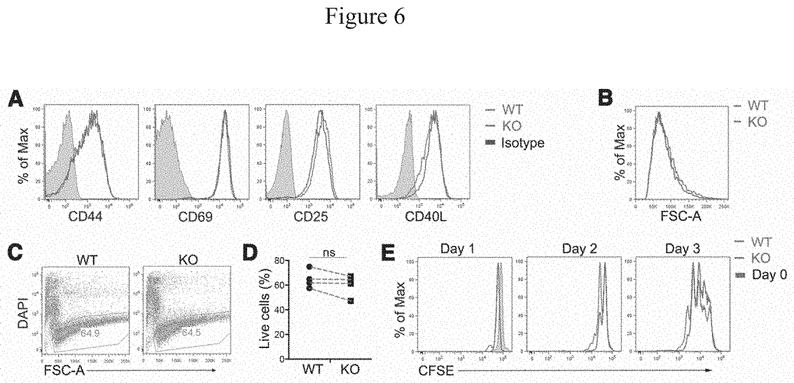

LDHA deficiency did not affect thymic development of conventional or regulatory T (Treg) cells (FIG. 4, panels A-C), or T cell homeostasis in peripheral lymph node and spleen (FIG. 5, panels A-G). LDHA deficiency also did not alter expression of activation markers, size or survival of activated CD4⁺ T cells (FIG. 6, panels A-D), while proliferation was slightly delayed (FIG. 6, panel E). These phenotypes were distinct from those of Glut1-deficient T cells (13), suggesting that general glucose metabolism, but not aerobic glycolysis, is required for T cell development and homeostasis.

### Example 2. LDHA Deficiency Results in Decreased IFN-γ Expression in T Cells

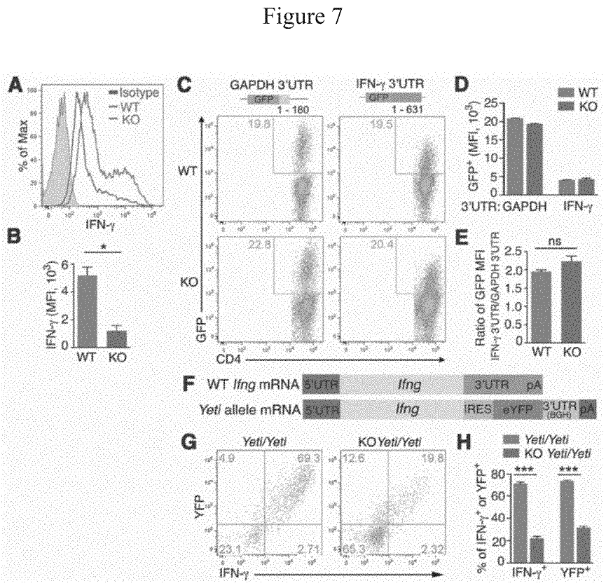

Glycolysis promotes expression of effector molecules including the type 1 cytokine IFN-γ (9, 12-15). Indeed, LDHA deficiency led to diminished IFN-γ expression in T cells differentiated under T helper 1 (Th1) conditions (FIG. 7, panels A and B). A recent study proposed that aerobic glycolysis enhanced IFN-γ production by sequestering GAPDH away from binding to the Ifng 3′UTR thereby enhancing IFN-γ translation (12). To determine whether such regulation accounted for reduced IFN-γ expression in KO T cells, we used a green florescent protein (GFP) reporter in which the GFP open reading frame was fused to the 3′UTR of Ifng or Gapdh, with the latter not known to repress mRNA translation (19). As previously reported (19), GFP expression controlled by the Ifng 3′UTR showed decreased mean fluorescence intensity compared to that regulated by the Gapdh 3′UTR in WT T cells (FIG. 7, panels C-E). Surprisingly, GFP expression under the control of Ifng 3′UTR was diminished to a similar extent in KO T cells (FIG. 7, panels C-E), suggesting that Ifng 3′UTR is insufficient to mediate aerobic glycolysis regulation of IFN-γ expression.

To explore the definitive function of 3′UTR in LDHA control of IFN-γ expression, we used an Ifng reporter allele Yeti (Yellow-enhanced transcript for IFN-γ) (20), in which the Ifng 3′UTR was replaced by 3′UTR of the bovine growth hormone (BGH) gene (FIG. 7, panel F). We found that expression of both IFN-γ and yellow fluorescent protein (YFP) driven by the internal ribosome entry site (IRES) element in the reporter allele were proportionally reduced in the absence of LDHA (FIG. 7, panels G and H), demonstrating that Ifng 3′UTR is not required for LDHA control of IFN-γ expression.

### Example 3. Histone Acetylation and Cytosolic Acetyl CoA are Reduced in LDHA-Deficient T Cells

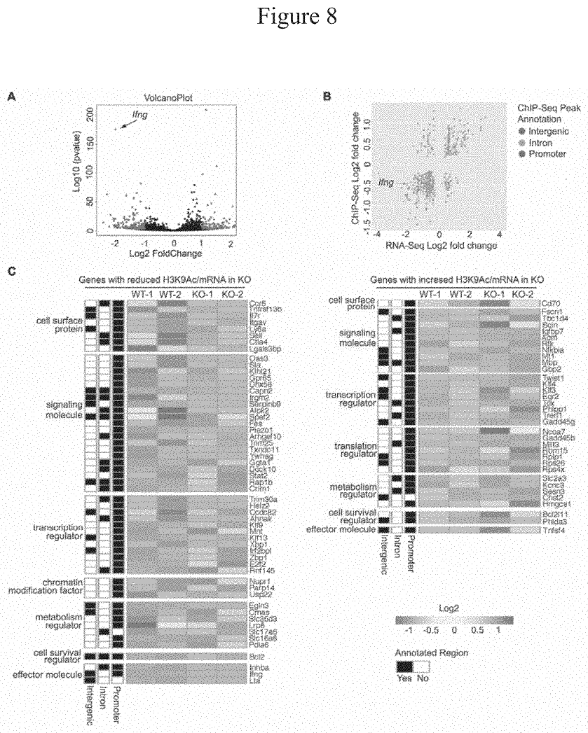

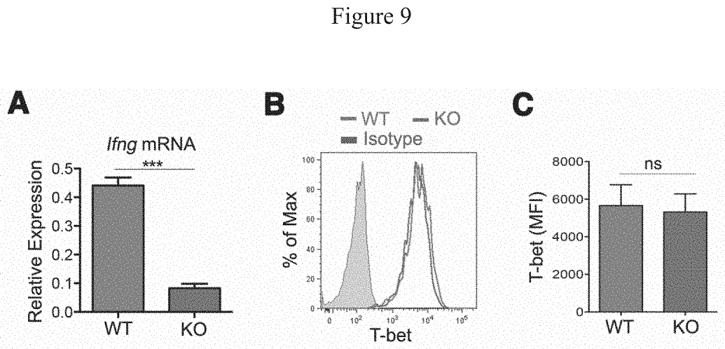

To determine whether reduced IFN-γ production in KO T cells was caused by diminished transcription, we performed RNA sequencing experiments. 363 transcripts were differentially expressed between WT and KO Th1 cells, among which 220 transcripts, including Ifng, were downregulated in KO T cells (FIG. 8, panel A, and FIG. 9, panel A). IFN-γ transcription in Th1 cells is induced by T-bet (21). However, T-bet expression was not affected in KO T cells (FIG. 9, panels B and C), suggesting that LDHA promotes IFN-γ expression via T-bet-independent mechanisms.

Glucose metabolism is implicated in the control of gene expression through epigenetic mechanisms including histone acetylation (7, 22). We performed chromatin immunoprecipitation-sequencing (ChIP-seq) analysis of histone H3 acetylation at the lysine 9 residue (H3K9Ac), a histone mark associated with active transcription. The ChIP-seq analysis showed that the differentially expressed genes between WT and KO Th1 cells had varying levels of H3K9Ac (FIG. 8, panel B), which encoded proteins involved in signal transduction, transcription, metabolism, and effector functions (FIG. 8, panel C). Notably, 86% downregulated transcripts including Ifng had decreased H3K9Ac in KO T cells (FIG. 8, panel B), suggesting that LDHA may promote IFN-γ expression by modulating histone acetylation.

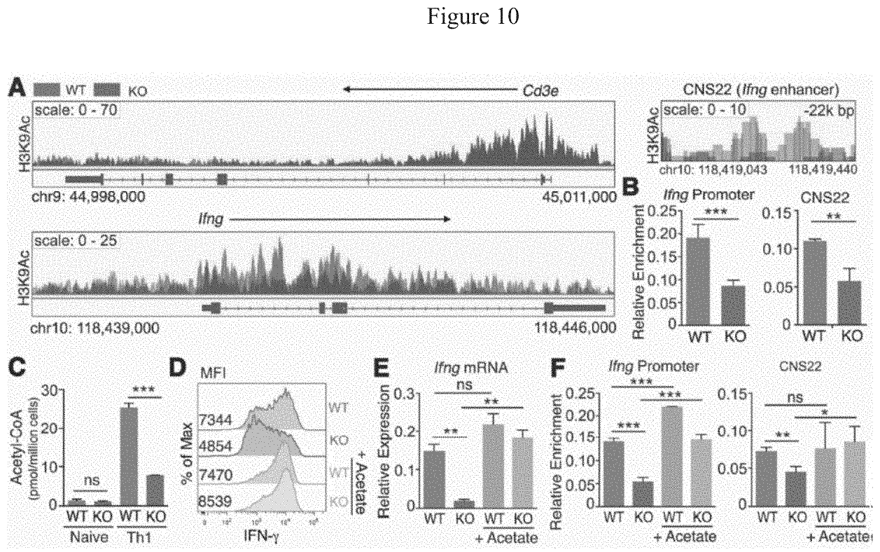

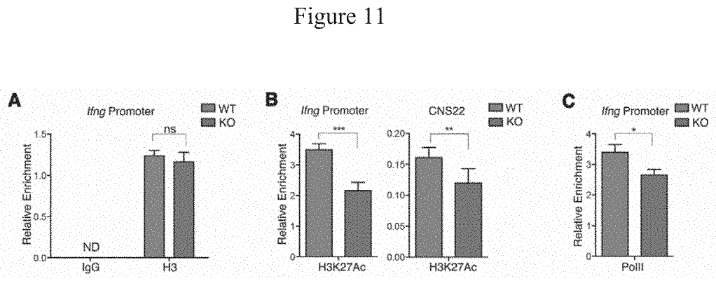

Compared to the constitutively active Cd3e locus, diminished H3K9Ac was observed in Ifng promoter, gene body and the conserved noncoding sequence 22 kilobase pairs upstream of Ifng (CNS-22) in KO T cells (FIG. 10, panels A and B). Acetylation of histone H3 at lysine 27 (H3K27Ac) was also reduced, while total histone H3 was comparable (FIG. 11, panels A and B). Importantly, diminished histone acetylation was associated with reduced RNA polymerase II (PolII) recruitment to the Ifng locus in KO T cells (FIG. 11, panel C). Histone acetylation requires acetyl coenzyme A (acetyl-CoA) as a substrate, with glucose being a critical source. Considering enhanced TCA cycle activity in KO T cells (FIG. 2, panels A-I and FIG. 3), we hypothesized that in the absence of LDHA, less citrate would be exported from the mitochondria for acetyl-CoA regeneration. Indeed, cytosolic acetyl-CoA was decreased in LDHA-deficient Th1 cells (FIG. 10, panel C).

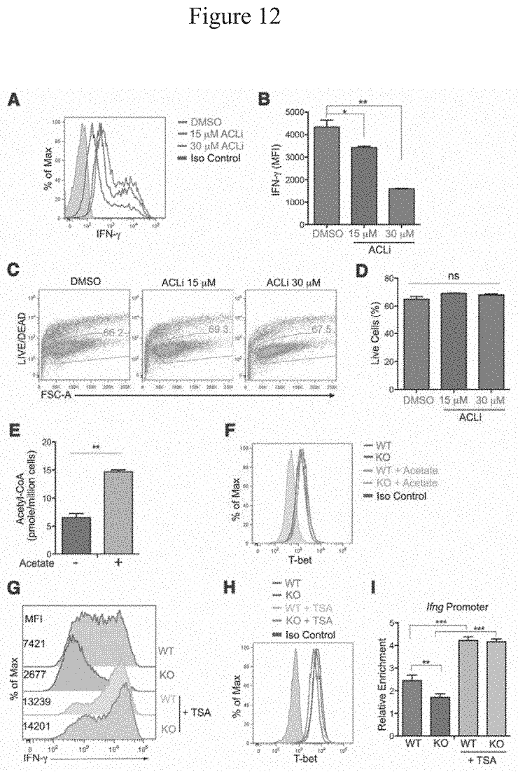

To determine whether reduced cytosolic acetyl-CoA was sufficient to repress IFN-γ expression, we inhibited ATP-citrate lyase (ACL), the enzyme converting citrate to acetyl-CoA, and found that IFN-γ expression was diminished in WT T cells (FIG. 12, panels A-D). Acetyl-CoA can be generated from acetate by acetyl-CoA synthetase, independent of citrate (23). Acetate supplementation augmented acetyl-CoA production (FIG. 12, panel E), and corrected IFN-γ expression in KO T cells (FIG. 10, panels D and E) without affecting T-bet expression (FIG. 12, panel F). Instead, enhanced IFN-γ production was associated with normalization of H3K9Ac in Ifng promoter and enhancer regions (FIG. 10, panel F). Histone acetylation is a dynamic process controlled by competing activities of histone acetyltransferases (HATs) and histone deacetylases (HDACs) (24). TSA, a HDAC inhibitor, rectified IFN-γ expression in KO T cells without affecting T-bet expression (FIG. 12, panels G and H), which was associated with increased H3K9Ac in the Ifng locus (FIG. 12, panel I). Collectively, these findings demonstrate that LDHA promotes IFN-γ expression through an epigenetic mechanism of histone acetylation.

### Example 4. LDHA Inhibition or Deficiency Inhibits IFN-γ Expression and Reduces T-Cell Mediated Autoinflammation

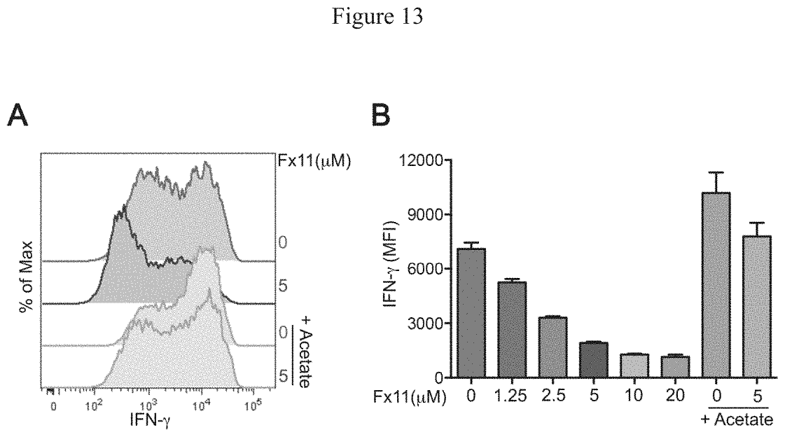

To test whether LDHA activity is required for its regulation of cytokine production, a LDHA inhibitor FX11 was used. We found that FX11 dose-dependently inhibited IFN-γ expression in T cells (FIG. 13, panels A-B). Importantly, the inhibitory effect of FX11 on IFN-γ expression was fully nullified by supplementing cells with acetate, supporting the conclusion that FX11 inhibits IFN-γ expression through modulation of acetyl-CoA levels. These data indicate that LDHA and ACL inhibitors can be useful in treatment of autoimmune/autoinflammatory diseases and rejection of transplant. Also, these inhibitors may be useful in treatment of any other diseases caused by excessive production of pro-inflammatory cytokines, such as sepsis shock in various infections (both bacterial and viral).

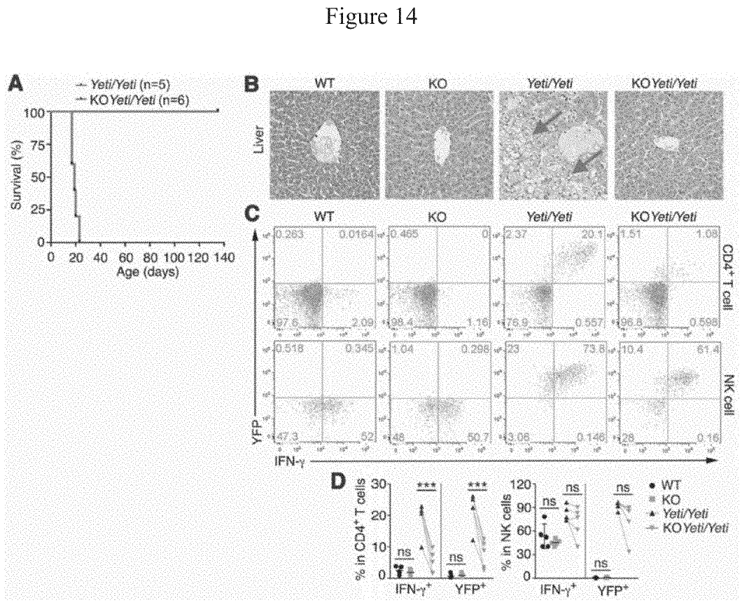

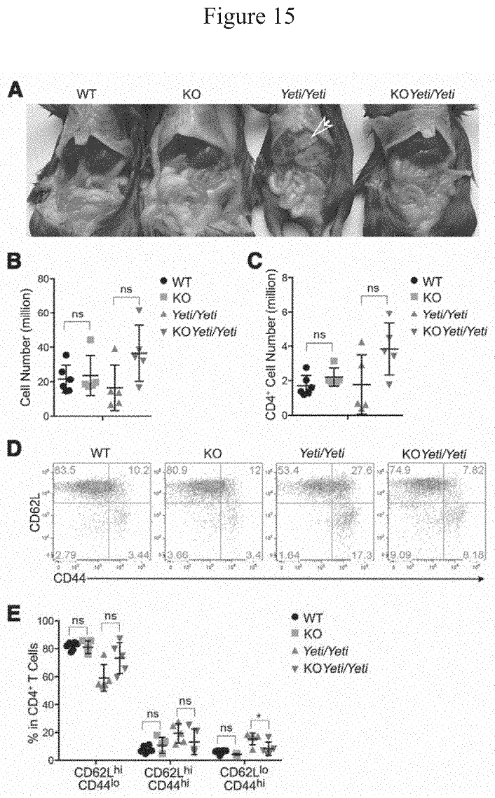

In the absence of endogenous 3′UTR, the Ifng transcript in Yeti/Yeti mice is stable, resulting in sustained IFN-γ production and a lethal autoinflammatory phenotype (25). Indeed, all Yeti/Yeti mice succumbed to death by 3 weeks of age (FIG. 14, panel A), which was associated with severe liver damage (FIG. 14, panel B, and FIG. 15, panel A). Deletion of Ldha in T cells corrected the liver immunopathology and conferred long-term survival of Yeti/Yeti mice (FIG. 14, panels A and B; FIG. 15, panel A). LDHA deficiency did not substantially affect the cellularity or activation of Yeti/Yeti CD4⁺ T cells (FIG. 15, panels B-E). However, IFN-γ expression in Yeti/Yeti CD4⁺ T cells was diminished in the absence of LDHA, while its expression in LDHA-sufficient Yeti/Yeti NK cells was not affected (FIG. 14, panels C and D). The correction of a lethal autoinflammatory disease in Yeti/Yeti mice by T cell-specific deletion of Ldha supports that aerobic glycolysis promotes IFN-γ expression in T cells through an epigenetic mechanism of Ifng transcription but not via a 3′UTR-dependent mechanism of translation in vivo.

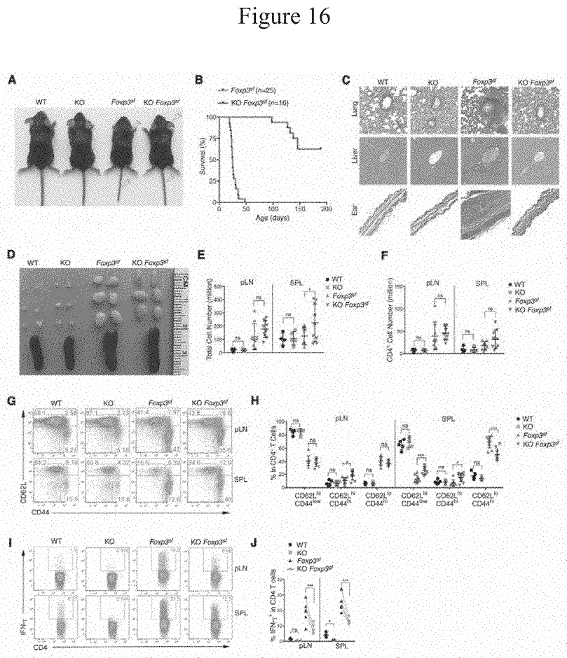

To further explore aerobic glycolysis in control of effector T cell responses, we used Scurfy mice with a mutation in the Treg cell lineage Foxp3 gene (Foxp3sf) (26). LDHA deficiency in T cells corrected the Scurfy phenotype and extended life span of Foxp3sf mice (FIG. 16, panels A and B), which was associated with reduced inflammation in multiple organs without affecting lymphadenopathy or splenomegaly (FIG. 16, panels C-F). T cell activation and expansion were comparable between Foxp3sf and KO Foxp3sf mice (FIG. 16, panels G and H), which was in line with our in vitro findings (FIG. 6, panels A-E). Nevertheless, CD4⁺ T cells produced less IFN-γ in the absence of LDHA (FIG. 16, panels I and J), demonstrating a critical role for LDHA-mediated aerobic glycolysis in promoting autoreactive Th1 cell responses.

### Example 5. LDHA Inhibition or Deficiency Inhibits IL-17A Expression

Naïve CD4 T cells (2.5×10⁴) were isolated from spleen and peripheral lymph nodes of Ldha^{fl/fl} (WT) and CD4^{Cre}Ldha^{fl/fl} (KO) mice. T cells were cultured with antigen-presenting cells (APCs) at a ratio of 10:1 (APC:T) in T-cell medium (RPMI-1640, 10% fetal bovine serum, 2 mM L-glutamine, 55 μM 2-mercaptoethanol, 100 U/mL penicillin, and 100 mg/mL streptomycin) under Th17 differentiation conditions (1 anti-CD3, 1 μg/mL anti-CD28, 2 ng/mL TGF-β, 10 ng/mL IL-6, and 10 μg/L α-IL2). WT cells were treated with 0.3 μM, 10 μM, or 30 μM GNE-140, or were untreated. Cultured cells were harvested on day 4, and were stimulated with ionomycin and PMA for 4 hours. Cytokines and transcription factors were analyzed by flow cytometry.

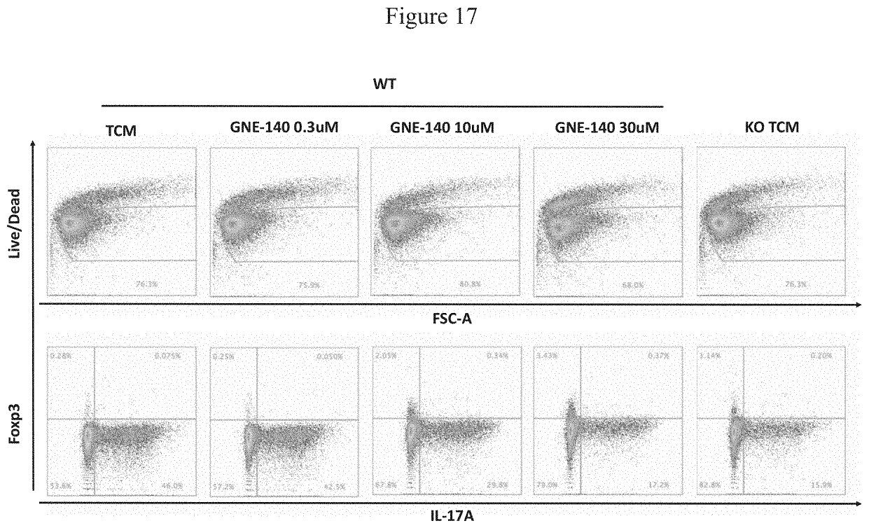

Under untreated conditions, KO T cells produced a significantly lower amount of IL-17A than WT T cells (FIG. 17). GNE-140 treatment inhibited IL-17A expression in WT T cells in a dose-dependent manner. At a concentration of 30 μM, WT T cells produced a similar level of IL-17A as KO T cells.

### Example 6. Inhibition of ACL is Protective in a Mouse Model of Multiple Sclerosis

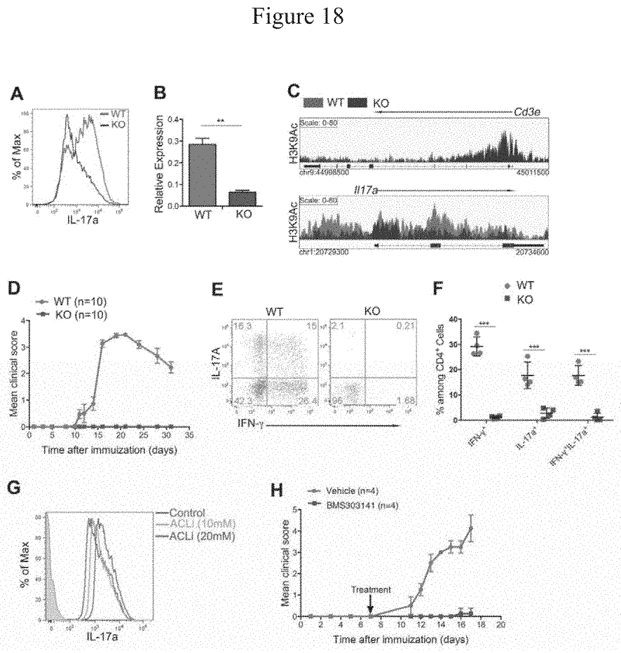

The same mechanism that we proposed for LDHA regulation of Th1 cell differentiation also operates in Th17 cells. We found that LDHA-deficiency resulted in diminished IL-17a production under Th17 culture conditions (FIG. 18, panels A and B), which was associated with reduced histone acetylation at Il17a locus (FIG. 18, panel C). Th17 cells play a critical role in experimental autoimmune encephalomyelitis (EAE), a mouse model of the human multiple sclerosis disease. Remarkably, LDHA deficiency in T cells completely protected mice from EAE (FIG. 18, panel D), which was associated with reduced production of both IL-17a and IFN-γ from CD4⁺ T cells isolated from spinal cord and cerebellum (FIG. 18, panels E and F).

To test whether reduced acetyl-CoA production was sufficient to blunt Th17 response, we added an ACL inhibitor (BMS 303141) to the Th17 culture, and found that it repressed IL-17a production (FIG. 18, panel G). Remarkably, in vivo treatment of mice with BMS 303141 completely protected mice from EAE (FIG. 18, panel H), indicating that ACL inhibition can be used to treat multiple sclerosis.

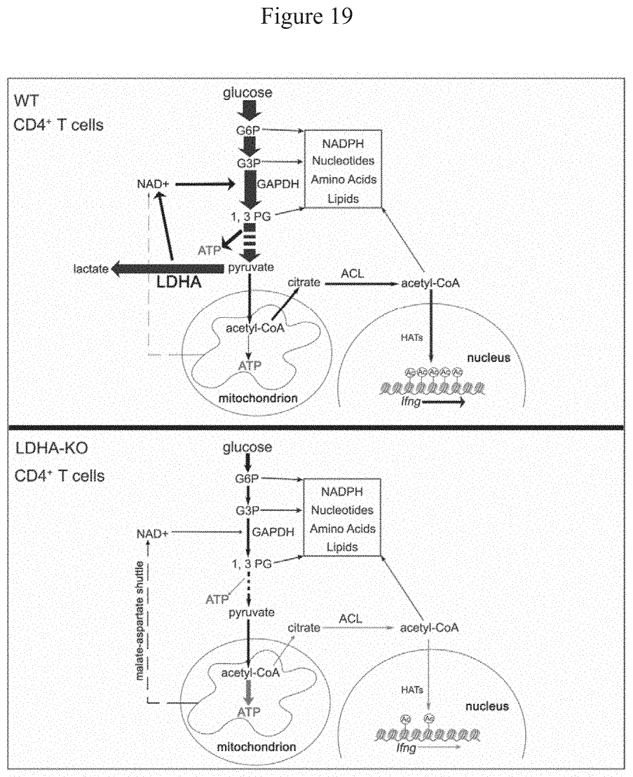

Overall, our findings in Examples 1-6 do not support a translational mechanism of aerobic glycolysis in IFN-γ production (12), as LDHA promotes IFN-γ expression independent of its 3′UTR. Previous studies have shown that utilization of glycolytic intermediates for biosynthesis accounts for a small fraction (˜7%) of the glucose consumed in activated T cells (27), while aerobic glycolysis produces the majority (˜60%) of ATP (28). Hence, LDHA-mediated aerobic glycolysis may primarily relieve the burden of mitochondria as an energy house to ‘burn’ carbons to generate ATP. As a result, more citrate can be exported out of mitochondria to generate acetyl-CoA and promote histone acetylation in selected gene loci (FIG. 19). LDHA inhibitors are being developed to target tumor cell metabolism (29). Our data suggest that LDHA inhibitors could be immunosuppressive, and thus may complicate their applications in cancer. Instead, LDHA inhibitors, as well as inhibitors targeting other enzymes involved in acetyl-CoA metabolism, including ACL, may be useful in treatment of T-cell mediated immune diseases, such as autoimmunity and transplant rejection.

### Example 7. Materials and Methods

### Mice

Mice with one targeted allele of Ldha (Ldha (Ldha^{tm1a(EUCOMM)Wtsi}) were obtained from EUCOMM, in which the third exon was flanked by two loxp sites. The mice were first crossed with a transgenic Flipase strain (Jackson Laboratory) to remove the Neo cassette, and then crossed with CD4^{Cre} transgenic mice to specifically delete Ldha in T cells. Yeti mice were kindly provided by Dr. Richard Locksley (UCSF). C57BL/6, CD4^{Cre and Foxp}3^{sf} mice were purchased from the Jackson Laboratory. All mice were maintained in the MSKCC animal facility under SPF conditions, and animal experimentation was conducted in accordance with institutional guidelines.

### Antibodies

Anti-LDHA (2012S), anti-GAPDH (D16H11), anti-Histone 3 (D2B12), and normal rabbit IgG were purchased from Cell Signaling. Anti-LDHB (60H11), anti-Histone H3 (acetyl K9) (ab4441), anti-Histone H3 (acetyl K27) (ab4729), and anti-RNA polymerase II CTD repeat YSPTSPS (ab5131) were obtained from Abcam. Fluorescent-dye-labeled antibodies against CD4, CD8, TCR-β, CD44, CD62L, CD69, CD25, CD40L, IFN-γ, NK1.1, Foxp3, and T-bet were purchased from eBiosciences, BioLegend, or Tonbo. Blocking antibodies for FcγR (2.4G2) and IL-4 (11B11), as well as anti-CD3 (145-2C11) and anti-CD28 (37.51) were obtained from Bio X Cell.

### Flow Cytometry

Cells from thymi, spleens, and lymph nodes were depleted of erythrocytes by hypotonic lysis. Cells were incubated with specific antibodies for 15 min on ice in the presence of anti-FcγR to block FcγR binding. Dead cells were excluded by DAPI (Invitrogen) staining. To determine cytokine expression, cells were stimulated with 50 ng/mL phorbol 12-myristate 13-acetate (Sigma) and 1 μM ionomycin (Sigma) in the presence of GolgiStop (BD Biosciences) for 4 h. After stimulation, cells were stained with cell-surface marker antibodies and LIVE/DEAD Fixable dye (Invitrogen) to exclude dead cells, fixed, and permeabilized with a transcription factor staining kit (eBioscience), followed by staining with cytokine antibodies. All samples were acquired with an LSR II flow cytometer (Becton, Dickinson) and analyzed with FlowJo software (TreeStar).

### In Vitro Cell Culture

CD62L⁺CD44⁻CD25⁻CD4⁺ T cells were isolated from spleen and peripheral lymph nodes of wild-type (WT) or CD4^{Cre}Ldha^{fl/fl} (KO) mice with naïve CD4⁺ T cells isolation kits (Miltenyi or Stem Cell). Purity was checked by flow cytometry, and was over 98%. For in vitro T cell activation, 0.2-0.5×10⁶ naïve CD4⁺ T cells were cultured on a 24-well plate pre-coated with 5 μg/mL anti-CD3 in T cell medium [RPMI/1640, 10% fetal bovine serum, 2 mM glutamine, 1 mM sodium pyruvate, 1× non-essential amino acid (Invitrogen), 10 mM HEPES, 55 μM 2-mercaptoethanol, 100 U/ml penicillin, 100 mg/ml streptomycin] supplemented with 2 μg/mL anti-CD28 and 100 U/ml IL-2. For T helper 1 (Th1) cell differentiation, T cells were cultured with the addition of 10 μg/mL anti-IL-4 and 10 ng/mL IL-12. For T helper 17 (Th17) cell differentiation, T cells were cultured with the addition of 10 μg/mL anti-IL-4, 10 μg/mL anti-IFN-γ, 10 μg/mL anti-IL-2, 2 ng/ml TGF-β, 10 ng/ml IL-6, 10 ng/ml IL-23, and 10 ng/mL IL-113. For cell proliferation experiments, naïve CD4⁺ T cells were labeled with CFSE (Invitrogen), and activated on a 24-well plate, pre-coated with 5 μg/mL anti-CD3, in T cell culture medium supplemented with 2 μg/mL anti-CD28 and 100 U/mL IL-2. CFSE dilution was assessed by flow cytometry on days 1, 2, and 3 post-activation. For sodium acetate and TSA treatment experiment, cells were differentiated under Th1 conditions for 3 days, and cultured in T cell medium supplemented with 100 U/mL IL-2, 10 μg/mL anti-IL-4, 10 ng/mL IL-12, and the indicated concentrations of sodium acetate (Sigma), TSA (Promega), or vesicle for 24 h. For ATP citrate lyase (ACL) inhibitor experiments, naïve CD4⁺ T cells isolated from WT mice were cultured under Th1 conditions for 2 days or under Th17 conditions for 3 days. DMSO and indicated concentrations of ATP citrate lyase inhibitor (BMS-303141, Sigma) were added into the culture for another 24 hours. For LDHA inhibitor experiments, naïve CD4⁺ T cells isolated from WT mice were cultured under Th1 or Th17 conditions for 3-4 days. DMSO and indicated concentrations of FX11 or GNE-140 were added into the culture for another 24 hours.

### 3′ UTR GFP Reporter Assay

3′UTR vectors were co-transfected with pCL-Eco helper plasmid into Phoenix cells, and the culture supernatant was used to infect activated T cells. Naïve CD4⁺ T cells isolated from WT and KO mice were activated with plated-bound anti-CD3 and soluble anti-CD28 for 2 days, and transduced with retroviral supernatants via spin-infection (2600 rpm for 2 h at 35° C.) and cultured for another 2 days. The GFP signal was examined by flow cytometry.

### Zymography for LDH Isoenzymes

Heart and muscle tissues were harvested from mice perfused with ice-cold PBS, and homogenized to single cells with non-denaturing cell lysis buffer (Cell Signaling). Activated CD4⁺ T cells and tissues were lysed with non-denaturing cell lysis buffer on ice for 20 min, and the cell lysate was centrifuged at 20,000 g for 10 min at 4° C. The supernatants were diluted with 2X native loading buffer (Biorad), and subjected to electrophoresis with precast 4-20% native gels (Biorad). The gels were subsequently incubated in a development solution containing 100 mM Tris-HCl (pH 8.0), 0.3 mM NAD (Sigma), 0.1 M sodium lactate (Sigma), 0.5 mM phenazin-methosulphate (Sigma) and 0.4 mM tetrazolium-blue (Sigma) at 37° C. for 30 min.

### Immunoblotting

Cells were lysed with a cell lysis buffer (Cell Signaling), and protein concentrations were determined with a BCA kit (Thermo Scientific). Protein extracts were separated by SDS-PAGE gels and transferred to nitrocellulose blotting membranes (GE Healthcare). The membranes were probed with antibodies and visualized with the Immobilon Western Chemiluminescent HRP Substrate (Millipore).

### Metabolic Assays

To determine glucose consumption and lactate production of activated CD4⁺ T cells, culture medium was replaced with fresh medium 48 h after T cell activation, and collected 24 h later. Medium alone in wells of the same plate was used as control. Glucose and lactate levels in culture media were measured with a glucose assay kit II (BioVison) and lactate assay kit II (Sigma).

For measurement of cytosolic acetyl-CoA, naïve CD4⁺ T cells were differentiated under Th1 conditions for 2 days and then cultured in fresh T cell medium for another 24 h. Cells were lysed with lysis buffer (1% Triton X-100, 20 mM Tris-HCl, pH=7.4, 150 mM NaCl) on ice for 10 min. The lysates were spun at 20, 000 g for 10 min at 4° C., the pellets (nuclei and heavy membrane) were discarded, and the supernatants were used for acetyl-CoA measurement with an Acetyl-CoA Assay Kit (Sigma). Freshly isolated naïve WT and KO CD4⁺ T cells were rested in T cell medium with 10 ng/mL IL-7 for 4 h at 37° C. in the incubator, and the level of acetyl-CoA was measured.

OCR and ECAR were measured with an XF96 extracellular flux analyzer (Seahorse Bioscience), following protocols recommended by the manufacturer. Briefly, freshly isolated CD4⁺ naïve or activated cells were seeded on XF96 microplates (150,000 cells/well) that had been pre-coated with Cell-Tak adhesive (BD Biosciences). The plates were quickly centrifuged to immobilize cells. Cells were maintained in a non-buffered assay medium (Seahorse Biosciences) in a non-CO₂ incubator for 30 min before the assay. Glycolysis was measured with XF glycolysis stress test kit (Seahorse Biosciences). Initially, cells were incubated in the glycolysis stress test medium without glucose, and ECAR was assessed. Three baseline recordings were made, followed by sequential injection of 10 mM glucose and 1 μM oligomycin, which inhibited mitochondrial ATP production and shifted the energy production to glycolysis. The increased ECAR revealed the maximum glycolytic capacity of T cells. The final injection was 100 mM 2-DG, a glucose analog that inhibited glycolysis. The resulting decrease of ECAR confirmed that the ECAR observed in experiments was due to glycolysis. The Mito stress test kit (Seahorse Biosciences) was used to test OCR under different conditions. Three baseline recordings were made, followed by sequential injection of 1 μM oligomycin, 0.25 μM FCCP, which uncoupled oxygen consumption from ATP production to obtain maximal OCR, and 0.5 μM rotenone/antimycin A, which inhibited complex I and III.

Metabolic flux analysis of ¹³C₆-glucose in Th1 cells was determined by CE-MS. Briefly, ˜10 million WT or KO Th1 cells were labeled with ¹³C₆-glucose (10 mM) in glucose-deficient RPMI1640 supplemented with 10% dialyzed FBS and 100 U/mL IL-2 for 2 h. Metabolites were extracted according to the Floating Cells Protocol E-150782, and shipped to Human Metabolome Technologies Inc. (Boston) for CE-MS analysis (F-SCOPE). Data were from three biological replicates.

### RNA-Seq and Quantitative RT-PCR

Total RNA was extracted with RNeasy kit (QIAGEN), and cDNA libraries were generated and sequenced using a HiSeq 2000 platform (Illumina) at the Integrated Genomics Operation of MSKCC. For quantitative RT-PCR, RNA was reverse transcribed with Superscript III (Invitrogen). mRNA levels were normalized to β-Actin. The primers used were: Actin-F: 5′-GGCACCACACCTTCTACAATG-3′ (SEQ ID NO: 1); Actin-R: 5′-GTGGTGGTGAAGCTGTAGCC-3′ (SEQ ID NO: 2). Ifng-F: 5′-CTTTGGACCCTCTGACTTGAG-3′ (SEQ ID NO: 3), Ifng-R: 5′-TTCCACATCTATGCCACTTGAG-3′ (SEQ ID NO: 4).

### RNA-Seq Analysis

Quality Control of raw reads was done using FastQC (v0.11.2) (10) to ensure that there were no major flaws in sequencing. They were then mapped to mm 10 genome using STAR (v2.3.0e_r291) (30) and default parameters. The mapped reads were counted using htseq-count (v0.6.0, parameters -t exon) (31) and gene models from Ensembl (Mus_musculus.GRCm38.75.gtf). Differential expression was performed using DESeq2 (v1.2.10, default parameters) (32). Human orthologs were retrieved from Ensembl v75. DESeq2 normalized counts with human orthologs were provided to GSEA (v2-2.0.14) (33) for gene set enrichment analysis.

### ChIP-Seq and ChIP-qPCR

Chromatin immunoprecipitations were performed with SimpleChIP Enzymatic Chromatin IP Kit (Cell Signaling) according to manufacturer's instructions. Briefly, naïve CD4⁺ T cells were differentiated to Th1 cells for 3 days, and were fixed for 10 min at 25° C. with 1% formaldehyde. After incubation, glycine was added to a final concentration of 0.125 M to quench formaldehyde. Subsequently, cells were lysed, and chromatin was harvested and fragmented using enzymatic digestion, followed by sonication. The chromatin was then subjected to immunoprecipitation with anti-H3K9Ac at 4° C. overnight, and was incubated with protein G magnetic beads at 4° C. for 2 h. The immune complexes were washed and eluted in 150 μL elution buffer. Elute DNA and input DNA were incubated at 65° C. to reverse the crosslinking. After digestion with proteinase K, DNA was purified with spin columns. The library was prepared with KAPA library preparation kit (Kapa Biosystems) and sequenced using a HiSeq 2000 platform (Illumina) at the Integrated Genomics Operation of MSKCC. The relative abundance of precipitated DNA fragments was analyzed by qPCR using Power SYBR Green PCR Master Mix (Qiagen) and the enrichments were normalized to Cd3e promoter. The following primers were used for qPCR: Cd3e promoter-F: 5′-TCAGTGTGGAGGTGCTTTG-3′ (SEQ ID NO: 5), Cd3e promoter-R: 5′-CAGCCTTCCCATAAGGATGAA-3′ (SEQ ID NO: 6); Ifng promoter-F: 5′-GGAGCCTTCGATCAGGTATAAA-3′ (SEQ ID NO: 7), Ifng promoter-R: 5′-CTCAAGTCAGAGGGTCCAAAG-3′ (SEQ ID NO: 8); CNS22-F: 5′-GAGGCCAAATTTCTGCTCATTG-3′ (SEQ ID NO: 9), CNS22-R: 5′-GTTCTTTCAGGAAGCCCGTTA-3′ (SEQ ID NO: 10).

### ChIP-Seq Analysis

Reads were first trimmed using Trimmomatic (v0.33) (34). Reads were trimmed if first/last 3 nucleotides had phred quality score of <15, or at the point where a sliding window of 4 nucleotides averaged a phred quality score <15. Illumina adapters were also removed. The reads were then aligned using bowtie2 (v 2.2.6, options -fr --no-discordant) (35). Multimapping reads were removed after alignment. Peak calling was done using MACS2 (v2.1.0) (36) on pooled replicates and individual samples using p-value cutoff of 0.01. The peaks were then filtered further using IDR (37) to make sure the peaks were consistent among replicates. The promoter was annotated as region within 2, 000 bp from TSS, intron was annotated as region within the gene body but not in the promoter region, and peaks at regions outside but close to gene body were annotated as intergenic.

RNA-seq and ChIP-Seq data are deposited in the Genome Expression Omnibus under accession number GSE86188, the content of which is incorporated herein by reference.

### Histopathology

Tissues from euthanized animals were fixed in Safefix II (Fisher) and embedded in paraffin. 5-μm sections were stained with haematoxylin and eosin.

### Statistical Analysis

Statistical tests were performed with Prism (GraphPad). A value of p<0.05 were considered statistically significant. All error bars represent standard error (SD).

### REFERENCES

1. R. Wang, D. R. Green, Metabolic checkpoints in activated T cells. Nat Immunol 13, 907-915 (2012).
2. E. L. Pearce, M. C. Poffenberger, C. H. Chang, R. G. Jones, Fueling immunity: insights into metabolism and lymphocyte function. Science 342, 1242454 (2013).
3. N. J. MacIver, R. D. Michalek, J. C. Rathmell, Metabolic regulation of T lymphocytes. Annu Rev Immunol 31, 259-283 (2013).
4. K. Ganeshan, A. Chawla, Metabolic regulation of immune responses. Annu Rev Immunol 32, 609-634 (2014).
5. K. A. Frauwirth, J. L. Riley, M. H. Harris, R. V. Parry, J. C. Rathmell, D. R. Plas, R. L. Elstrom, C. H. June, C. B. Thompson, The CD28 signaling pathway regulates glucose metabolism. Immunity 16, 769-777 (2002).
6. M. G. Vander Heiden, L. C. Cantley, C. B. Thompson, Understanding the Warburg effect: the metabolic requirements of cell proliferation. Science 324, 1029-1033 (2009).
7. C. Lu, C. B. Thompson, Metabolic regulation of epigenetics. Cell Metab 16, 9-17 (2012).
8. M. V. Liberti, J. W. Locasale, The Warburg Effect: How Does it Benefit Cancer Cells? Trends Biochem Sci 41, 211-218 (2016).
9. C. M. Cham, T. F. Gajewski, Glucose availability regulates IFN-gamma production and p70S6 kinase activation in CD8(+) effector T cells. J Immunol 174, 4670-4677 (2005).
10. R. N. Wang, C. P. Dillon, L. Z. Shi, S. Milasta, R. Carter, D. Finkelstein, L. L. McCormick, P. Fitzgerald, H. B. Chi, J. Munger, D. R. Green, The Transcription Factor Myc Controls Metabolic Reprogramming upon T Lymphocyte Activation. Immunity 35, 871-882 (2011).
11. L. Z. Shi, R. Wang, G. Huang, P. Vogel, G. Neale, D. R. Green, H. Chi, HlFlalpha-dependent glycolytic pathway orchestrates a metabolic checkpoint for the differentiation of TH17 and Treg cells. J Exp Med 208, 1367-1376 (2011).
12. C. H. Chang, J. D. Curtis, L. B. Maggi, Jr., B. Faubert, A. V. Villarino, D. O'Sullivan, S. C. Huang, G. J. van der Windt, J. Blagih, J. Qiu, J. D. Weber, E. J. Pearce, R. G. Jones, E. L. Pearce, Posttranscriptional control of T cell effector function by aerobic glycolysis. Cell 153, 1239-1251 (2013).
13. A. N. Macintyre, V. A. Gerriets, A. G. Nichols, R. D. Michalek, M. C. Rudolph, D. Deoliveira, S. M. Anderson, E. D. Abel, B. J. Chen, L. P. Hale, J. C. Rathmell, The Glucose Transporter Glut1 Is Selectively Essential for CD4 T Cell Activation and Effector Function. Cell Metabolism 20, 61-72 (2014).
14. J. Blagih, F. Coulombe, E. E. Vincent, F. Dupuy, G. Galicia-Vazquez, E. Yurchenko, T. C. Raissi, G. J. van der Windt, B. Viollet, E. L. Pearce, J. Pelletier, C. A. Piccirillo, C. M. Krawczyk, M. Divangahi, R. G. Jones, The energy sensor AMPK regulates T cell metabolic adaptation and effector responses in vivo. Immunity 42, 41-54 (2015).
15. P. C. Ho, J. D. Bihuniak, A. N. Macintyre, M. Staron, X. J. Liu, R. Amezquita, Y. C. Tsui, G. L. Cui, G. Micevic, J. C. Perales, S. H. Kleinstein, E. D. Abel, K. L. Insogna, S. Feske, J. W. Locasale, M. W. Bosenberg, J. C. Rathmell, S. M. Kaech, Phosphoenolpyruvate Is a Metabolic Checkpoint of Anti-tumor T Cell Responses. Cell 162, 1217-1228 (2015).
16. E. Bustamante, P. L. Pedersen, High aerobic glycolysis of rat hepatoma cells in culture: role of mitochondrial hexokinase. Proc Natl Acad Sci USA 74, 3735-3739 (1977).
17. J. J. L. Holbrook, A; Steindel, S. J.; Rossmann, M. G., 4 Lactate Dehydrogenase. The Enzymes 11, 191-292 (1975).
18. See Example 7.
19. A. V. Villarino, S. D. Katzman, E. Gallo, 0. Miller, S. Jiang, M. T. McManus, A. K. Abbas, Posttranscriptional silencing of effector cytokine mRNA underlies the anergic phenotype of self-reactive T cells. Immunity 34, 50-60 (2011).
20. D. B. Stetson, M. Mohrs, R. L. Reinhardt, J. L. Baron, Z. E. Wang, L. Gapin, M. Kronenberg, R. M. Locksley, Constitutive cytokine mRNAs mark natural killer (NK) and NK T cells poised for rapid effector function. J Exp Med 198, 1069-1076 (2003).
21. S. J. Szabo, S. T. Kim, G. L. Costa, X. Zhang, C. G. Fathman, L. H. Glimcher, A novel transcription factor, T-bet, directs Th1 lineage commitment. Cell 100, 655-669 (2000).
22. K. E. Wellen, G. Hatzivassiliou, U. M. Sachdeva, T. V. Bui, J. R. Cross, C. B. Thompson, ATP-citrate lyase links cellular metabolism to histone acetylation. Science 324, 1076-1080 (2009).
23. A. Luong, V. C. Hannah, M. S. Brown, J. L. Goldstein, Molecular characterization of human acetyl-CoA synthetase, an enzyme regulated by sterol regulatory element-binding proteins. J Biol Chem 275, 26458-26466 (2000).
24. E. Verdin, M. Ott, 50 years of protein acetylation: from gene regulation to epigenetics, metabolism and beyond. Nat Rev Mol Cell Biol 16, 258-264 (2015).
25. R. L. Reinhardt, H. E. Liang, K. Bao, A. E. Price, M. Mohrs, B. L. Kelly, R. M. Locksley, A novel model for IFN-gamma-mediated autoinflammatory syndromes. J Immunol 194, 2358-2368 (2015).
26. M. E. Brunkow, E. W. Jeffery, K. A. Hjerrild, B. Paeper, L. B. Clark, S. A. Yasayko, J. E. Wilkinson, D. Galas, S. F. Ziegler, F. Ramsdell, Disruption of a new forkhead/winged-helix protein, scurfin, results in the fatal lymphoproliferative disorder of the scurfy mouse. Nat Genet 27, 68-73 (2001).
27. D. A. Hume, J. L. Radik, E. Ferber, M. J. Weidemann, Aerobic Glycolysis and Lymphocyte-Transformation. Biochem J 174, 703-709 (1978).
28. M. Guppy, E. Greiner, K. Brand, The Role of the Crabtree Effect and an Endogenous Fuel in the Energy-Metabolism of Resting and Proliferating Thymocytes. Eur J Biochem 212, 95-99 (1993).
29. K. Augoff, A. Hryniewicz-Jankowska, R. Tabola, Lactate dehydrogenase 5: An old friend and a new hope in the war on cancer. Cancer Lett 358, 1-7 (2015).
30. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics 29, 15-21 (2013).
31. S. Anders, P. T. Pyl, W. Huber, HTSeq-a Python framework to work with high-throughput sequencing data. Bioinformatics 31, 166-169 (2015).
32. M. I. Love, W. Huber, S. Anders, Moderated estimation of fold change and dispersion for RNA-seq data with DESeq2. Genome Biol 15, (2014).
33. A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: a knowledge-based approach for interpreting genome-wide expression profiles. Proc Natl Acad Sci USA 102, 15545-15550 (2005).
34. A. M. Bolger, M. Lohse, B. Usadel, Trimmomatic: a flexible trimmer for Illumina sequence data. Bioinformatics 30, 2114-2120 (2014).
35. B. Langmead, S. L. Salzberg, Fast gapped-read alignment with Bowtie 2. Nat Methods 9, 357-359 (2012).
36. Y. Zhang, T. Liu, C. A. Meyer, J. Eeckhoute, D. S. Johnson, B. E. Bernstein, C. Nusbaum, R. M. Myers, M. Brown, W. Li, X. S. Liu, Model-based analysis of ChIP-Seq (MACS). Genome Biol 9, R137 (2008).
37. Q. H. Li, J. B. Brown, H. Y. Huang, P. J. Bickel, Measuring Reproducibility of High-Throughput Experiments. Ann Appl Stat 5, 1752-1779 (2011).
38. J. Billiard, J. B. Dennison, J. Briand, R. S. Annan, D. Chai, M. Colon, C. S. Dodson, S. A. Gilbert, J. Greshock, J. Jing, H. Lu, J. E. McSurdy-Freed, L. A. Orband-Miller, G. B. Mills, C. J. Quinn, J. L. Schneck, G. F. Scott, A. N. Shaw, G. M. Waitt, R. F. Wooster, K. J. Duffy, Quinoline 3-sulfonamides inhibit lactate dehydrogenase A and reverse aerobic glycolysis in cancer cells. Cancer Metab. 1, 19 (2013).

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims.

1. A method of treating or preventing a T cell-mediated disease or disorder, the method comprising administering to a subject having or susceptible to developing a T cell-mediated disease or disorder a composition comprising an effective amount of a lactate dehydrogenase A (LDHA) inhibitor.

2. The method of claim 1, wherein the LDHA inhibitor is selected from the group consisting of FX11, Galloflavin, GNE-140, GSK 2837808A, and NHI 2.

3. The method of claim 2, wherein the LDHA inhibitor is FX11.

4. The method of claim 1, wherein the composition is administered orally.

5. The method of claim 1, wherein the composition is administered intravenously.

6. The method of claim 1, wherein the subject is a human.

7. The method of claim 1, wherein the T cell-mediated disease or disorder is an autoimmune disorder, graft rejection, inflammation, or organ rejection.

8. The method of claim 7, wherein the autoimmune disorder is selected from the group consisting of graft-versus-host disease (GVHD), inflammatory bowel disease (IBD), multiple sclerosis (MS), psoriasis, rheumatoid arthritis, systemic lupus erythematosus (SLE), and type-1 diabetes.

9. The method of claim 7, wherein the composition is administered prophylactically within about 24 hours of an organ transplant or tissue graft.
